(19) United States
(12) Patent Application Publication
(10) Pub. No.: US 2017/0224526 A1
(43) Pub. Date: Aug. 10, 2017

(54) DEVICE FOR COLD OR HOT THERMAL STIMULATION AND METHOD FOR CONTROLLING AND ADJUSTING SAME

(71) Applicants: UNIVERSITE DE STRASBOURG, Strasbourg (FR); CENTRE NATIONAL DE LA RECHERCHE SCIENTIFIQUE, Strasbourg (FR)

(72) Inventors: André Dufour, Lampertheim (FR); Thierry Pebayle, Wolfisheim (FR)

(21) Appl. No.: 15/322,216

(22) PCT Filed: Jun. 25, 2015
(86) PCT No.: PCT/FR2015/051715

§ 371 Date: Apr. 10, 2017

(30) Foreign Application Priority Data
Jun. 27, 2014 (FR) 1456033
Mar. 12, 2015 (FR) 1552054

(51) Int. Cl. A61F 7/00

(52) U.S. Cl. CPC A61F 7/007; A61F 2007/0075; A61F 2007/0096; A61F 2007/0086

(57) ABSTRACT

The present invention relates to a control and command method for a device for thermal stimulation of animal or human tissue (1), said method being characterised in that it includes: determining a neutral temperature applied to the tissue (1), thermally adjusting the neutral temperature by activating a control loop, determining a cold or hot thermal stimulation temperature to be applied to the tissue (1), in alternation with the neutral temperature; determining a duration and a frequency of the thermal stimulations; in the case of an instruction to initiate the stimulation temperature by activating a control loop and by deactivating the control loop on the neutral temperature; and synchronising a physiological recording with the thermal stimulation.

### TECHNICAL DOMAIN

This invention refers to the general technical domain of the exploration of receivers and fibres in the peripheral nerve system, as part of fundamental research or clinical examination. Exploration like this is carried out by tactile stimulation, more specifically a pressure, by electrical stimulation or by thermal stimulation.

For instance, small diameter myelinised fibres only respond to cold or painful stimulations (electric or heat). Since it may be desirable to avoid painful stimulation during the investigation of these fibres, there stimulation by cold is particularly suitable. Electrophysiological investigation, for instance, electromyography or electroencephalography, requires very short stimulation times on the order of a few tens of milliseconds.

From the clinical standpoint, stimulations by cold are advantageous in neuropathic diagnosis of the small fibres involved in neuropathic pain. The systems evoking small fibre neuropathy are sensitive symptoms or symptoms of dysautonomy.

The invention also concerns the exploration of non-myelinised fibres, known as C fibres, by hot stimulations beneath the pain threshold, impossible with cold stimulations alone.

The invention also concerns the exploration of myelinised fibres, known as A Delta fibres, responding to painful hot stimulations because a cold stimulation activates the A Delta fibres without causing pain.

Therefore, the invention more specifically involves a thermal stimulation device and a test and control method controlling this thermal stimulation.

### STATE OF THE ART

Through document WO2004/103230A1, a hot and cold stimulation device is already known. This stimulation device comprises a block of thermal conducting material, held at a cold temperature of between 1° C. and 20° C. by means of a standard Peltier module. Between the patient's skin and this block, there is a heating film designed to maintain a neutral temperature at the point of contact with the skin. When the heating film is no longer powered, and accordingly is no longer heated, it is brought to the block to temperature by a simple thermal conduction. The skin is then stimulated. The major drawback of a thermal stimulation device like this is to be found in the thermal conduction speed of the materials used. A device of this type allows maximum cold stimulation speeds of around −40° C./s, considered very insufficient.

From the document US 2012/065713 there is also another known cold thermal stimulation device. This document describes the use of a thermal mass, a Peltier module and a thermal dissipator. In addition, the test and control method describes the use of a thermal block cooled by exposure to cold, on which a thermal stimulation device is arranged to obtain a sufficient temperature reduction to allow stimulations to be pursued.

Furthermore, the document WO 2012/154787 also describes a thermal stimulation device and a process. The device in question uses micro-Peltier components generating cold, in particular. The Peltier components in question are described in conjunction with a temperature sensor of the thermocouple type. However, it is noteworthy that this document describes very generally embodiments of a thermal stimulation device as well as various control methods in a relatively general approach, requiring the use of a heat transfer fluid to produce cold, a great disadvantage.

### DISCLOSURE OF THE INVENTION

The purpose of this invention, accordingly, is to overcome the drawbacks of the prior art and supply a novel thermal stimulation device that substantially increases the cooling speeds.

Another purpose of this invention aims at proposing a new more accurate and more reliable thermal stimulation device which is also more economical.

Another purpose of this invention aims at proposing a new thermal stimulation device capable of performing hot and cold stimulations in a particularly simple way.

Another purpose of this invention aims at proposing a new test and control method for a thermal stimulation device to explore the neural pathways with greater accuracy.

The purposes assigned to the invention are achieved by means of a cold or hot thermal stimulation device working on animal or human tissue, comprising a stimulation head designed to come into contact with the said tissue and a thermal control and regulation circuit for generating a stimulation temperature, characterised in that it comprises a thermal inertia mass, micro-Peltier components integral with one side of the thermal inertia mass to form a contact and with the tissue, a thermal dissipator, at least one Peltier module sandwiched between the thermal inertia mass and the thermal dissipator, at least one temperature sensor attached to a free face of a micro-Peltier component, the said sensor being connected to the control and regulation circuit, an electric power supply for the micro-Peltier components controlled by the control and regulation circuit to prepare their hot or cold surfaces in contact with the thermal inertia mass, the control and regulating circuit thus allowing a regulation loop to be performed to generate the cold or hot stimulation temperature, and an electric power supply for the Peltier module controlled by the control and regulation circuit to arrange its hot face in contact with the thermal dissipator or in contact with the thermal inertia mass, thus allowing a regulation loop to be performed for maintaining the thermal inertia mass at a neutral temperature by evacuating thermal energy drawn off from the thermal inertia mass or by compensating for the thermal energy drawn off, respectively during the cold and hot stimulation phases.

According to an example of an embodiment conforming to the invention, the temperature sensor is a thermocouple.

According to an example of an embodiment conforming to the invention, the control and regulating circuit includes a microcontroller with an interface for connection to a computer and an interface for connection to a physiological parameter recorder.

According to an example of an embodiment conforming to the invention, the micro-Peltier components, separate from each other and arranged in an array, are soldered to the end face of the thermal inertia mass, which is an electrical and thermal insulating material filling the free spaces located between the said micro-Peltier components.

According to another example of an embodiment conforming to the invention, the thermal stimulation device has an additional temperature sensor continuously reading the temperature of the inertia mass.

The purposes assigned to the invention are also achieved by means of inspection and control process for a thermal stimulation device of animal or human tissue, characterised in that it consists in:
e1) determining the neutral temperature applied to the tissue,
e2) carrying out thermal regulation of the neutral temperature by activating a regulating loop,
e3) determining a cold and/or hot thermal stimulation temperature to be applied to the tissue in alternation with the neutral temperature,
e4) determining a duration and a frequency for the cold or hot thermal stimulations,
e5) if an instruction is given to trigger cold or hot stimulation, carrying out regulation of the cold or hot thermal stimulation temperature by activating a regulating loop and deactivating the regulating loop controlling the neutral temperature,
e6) synchronising the triggering of a physiological parameter recorder with the initiating of cold or hot thermal stimulation, and
e7) reactivating the regulating loop controlling the neutral temperature after each cold or hot thermal stimulation.

According to an alternate embodiment conforming to the invention, the inspection and control process for a thermal stimulation device of animal or human tissue, is characterised in that it consists in:

e1) determining the neutral temperature applied to the tissue,
e2) carrying out thermal regulation of the neutral temperature by activating a regulating loop,
e3) determining a cold and/or hot thermal stimulation temperature to be applied to the tissue in alternation with the neutral temperature,
e4) determining a duration and a frequency for the cold or hot thermal stimulations,
e5) if an instruction is given to trigger cold or hot stimulation, carrying out regulation of the cold or hot thermal stimulation temperature by activating a regulating loop and deactivating the regulating loop controlling the neutral temperature,
e6) synchronising the triggering of a physiological parameter recorder with the triggering of cold or hot thermal stimulation.

According to an implementation example conforming to the invention, the method consists in using continuously controlling the measured temperatures.

According to an implementation example conforming to the invention, the method consists in using micro-Peltier components to generate the cold or hot thermal stimulation temperature.

According to an implementation example conforming to the invention, the method consists in using a thermal inertia mass and a thermal dissipator, combined with a Peltier module to generate the neutral temperature.

According to an implementation example conforming to the invention, the method consists in using the thermal inertia mass to evacuate thermal energy given off by the micro-Peltier components during the cold thermal stimulation phases.

In addition, the process consists in using the thermal inertia mass to compensate for the thermal energy drawn off by the micro-Peltier components during the hot thermal stimulation phases.

According to an implementation example conforming to the invention, the method consists in measuring the temperature of the tissue, at a determined frequency, and using the results of the measurement to determine or adjust the neutral temperature.

The thermal stimulation device according to the invention offers an enormous advantage of being able to produce cold temperatures in very short times, for instance with a preferential reduction of the temperature of around 300° C./s. A cooling rate like this allows the functional exploration of the “A delta” type nerve fibres or small diameter myelinised fibres involved in nociception and at the origin of many pathological conditions of the neural pathways. The size/power ratio of the micro-Peltier components is particularly suitable for obtaining such results.

Another advantage of the stimulating device according to the invention is the possibility of embedding in the stimulation head, in combination with means generating a cold thermal stimulation, a laser source to generate hot thermal stimulations for other functional explorations. An example of an embodiment like this makes it possible to obtain very small stimulation surfaces, for instance, less than one mm².

The stimulation device according to the invention is also particularly compact and can be manipulated with ease.

In addition, thanks to dissipation by the convecting of the heat generated by the stimulation head, there is no need for an air/water cooling system of the watercooling type. Furthermore, it is noteworthy that for cold production, the thermal stimulation device does not use a heat transfer fluid. This makes it possible for the stimulation device to be particularly simple and stable in its operation. No replacement of expendable products, such as a liquid or gas, is needed. The maintenance of the thermal stimulation device conforming to the invention is therefore particularly simple and economical.

In an outstanding manner, the stimulation device conforming to the invention offers a high thermal conductivity allowing very fast return to a neutral temperature by simple thermal conduction. Accordingly, the thermal stimulation device conforming to the invention allows the tissue to be exposed to cold and neutral temperatures, in alternation, and at a high frequency.

The thermal stimulation device conforming to the invention is also noteworthy in that it can be used to generate hot thermal stimulations with a fast temperature increase, preferably of 300° C./s. This is related to additional heating by a Joulian effect of the micro-Peltier components as a complement to the thermal-electric effect of the said micro-Peltier components during temperature increases.

By optimising various parameters, for instance the speed at which the electric current is generated in the micro-Peltier components, the nature of the soldered joints used, the component materials of the inertia mass, it is possible to achieve temperature increase and decrease rates of approximately 350° C./s or even 400° C./s in some cases.

Another advantage of the thermal stimulation device conforming to the invention is that the neutral temperature is very quickly re-established as soon as the electric supply of the micro-Peltier components is cut off, more especially because of the thinness and the low thermal inertia of the end coating in which the said micro-Peltier components are embedded. Accordingly, for cold or hot stimulation durations of approximately 100 ms, the cold stimulations can be repeated at a frequency of approximately 1 Hz, allowing the neutral temperature to be re-established after each cold or hot stimulation.

Another advantage of the invention is the fact that all the neural pathway investigation protocols, including alternating hot and cold stimulations, are accessible with one and the same thermal stimulating device conforming to the invention. There is no longer any need to change the application head or to use separate devices suited to specific protocols.

### BRIEF DESCRIPTION OF THE DRAWINGS

Other characteristics and advantages of this invention will appear more clearly from the reading of the following description, referring to the attached illustrations, given as nonlimiting examples in which:

FIG. 1 is a schematic view of an embodiment example of a thermal stimulation device conforming to the invention

FIG. 2 is a schematic top view of the free end of the stimulation head of the stimulation device shown in FIG. 1,

FIG. 3 is an example of the electronic architecture of a thermal a stimulation device conforming to the invention, and

FIG. 4 is a functional flowchart illustrating the steps of an example of the implementation of a test and control method conforming to the invention,

### INVENTION PRODUCTION METHOD(S)

The structurally and functionally identical components shown on several separate figures are given the same numerical or alphanumerical reference.

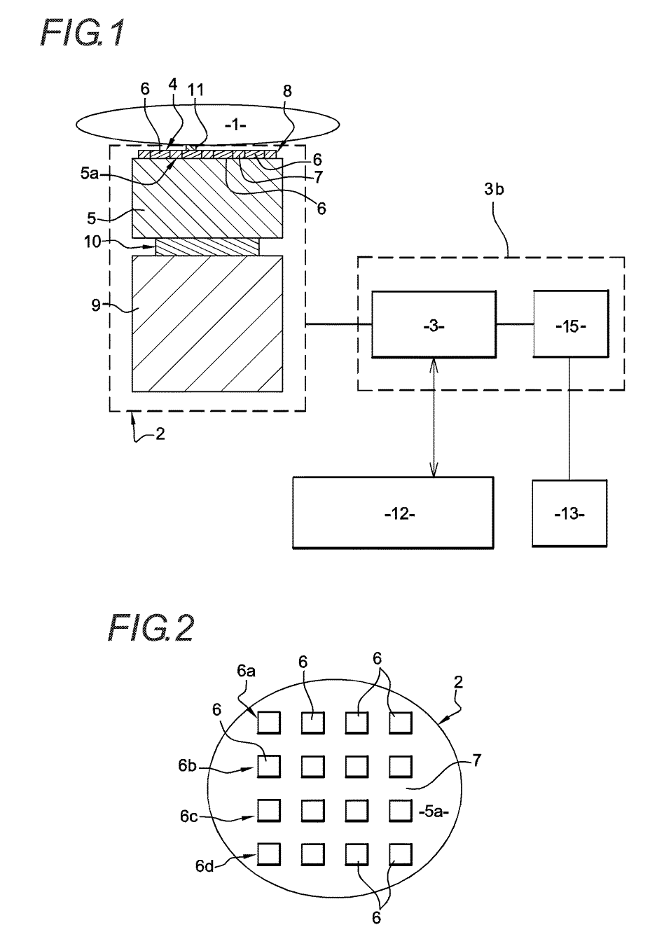

FIG. 1 is a schematic view of an embodiment example of a thermal stimulation device conforming to the invention. This thermal stimulation device allows the cold stimulation of animal or human tissue 1.

The thermal stimulation device conforming to the invention and described below, allows cold or hot thermal stimulation to be generated. Even if the generation of cold stimulations is described in greater detail below, the invention and especially the control and regulation method, also concern the generation of hot thermal stimulations or alternations of hot and cold thermal stimulations.

The thermal stimulation device includes a stimulation head 2 designed to come into contact with tissue 1.

The thermal stimulation device also includes a control and regulating circuit 3, generating for instance, a cold stimulation temperature at one of the ends 4 of stimulation head 2.

The stimulation head 2 comprises a thermal inertia mass 5 comprising, for instance, a block consisting of thermal conducting material. This block, weighing approximately 30 gr, is made, for instance, of copper or silver and, for instance, has a diameter of 25 mm and a thickness of 10 mm.

One end face 5a of the thermal inertia mass 5, located near the contact end 4 of the stimulation head 2, is covered with micro-Peltier components 6.

Advantageously, the micro-Peltier components 6 are made integral with the thermal inertia mass 5 by brazing.

For instance, the micro-Peltier components 6 shown in FIG. 2, are separated from one another in order to leave open spaces between the said micro-Peltier components 6, whose thickness is approximately 0.6 mm. These open spaces are advantageously filled with electric and thermal insulating material 7, such as an epoxy resin loaded with glass or phenol micro-balls. The micro-Peltier components 6, associated with insulating material 7 thus forms an end coat 8, designed to come into contact with tissue 1.

Therefore, advantageously, the end coat 8 has a thickness of approximately 0.6 mm and extremely low thermal inertia. The end coat 8 also has high thermal conductivity resulting from the component material of the micro-Peltier components 6 on the one hand, and its thinness on the other.

The distribution of the micro-Peltier components 6, of which there are for instance 16, is obtained by means of an array having four rows 6a, 6b, 6c, 6d and four columns.

The stimulation head 2 also includes a thermal dissipator 9. For instance, the latter is a passive thermal dissipator such as a high power LED dissipator. The thermal dissipator 9 can also be a natural or forced convection dissipator or a dissipator using water as a heat transfer fluid.

Each micro-Peltier component 6 has a cold active face measuring approximately 6 mm² and a hot active face of approximately 9 mm², used for brazing on the thermal inertia mass 5.

The array of micro-Peltier components 6, as shown in FIG. 2, advantageously has a power density in excess of 20 W/cm², and preferably of around 80 W/cm² or more.

The stimulation head 2 also comprises a standard Peltier module 10 sandwiched between the inertia mass 5 and the thermal dissipator 9. The Peltier module 10 comprises, for instance, a power density of approximately 10 W/cm².

The hot face of the Peltier module 10 is in contact with the thermal dissipator 9 and its cold face is in contact with the thermal inertia mass 5. The electric power supply of the Peltier module 10 can also be reversed so that its hot face is in contact with the thermal inertia mass 5 to ensure the thermal regulation of the said thermal inertia mass 5 under some conditions of use.

The Peltier module 10 and the thermal dissipator 9 also serve to evacuate the thermal energy of the thermal inertia mass 5 or to bring in thermal energy to the thermal inertia mass 5 to maintain it at a determined temperature.

The stimulation head 2 also comprises at least one temperature sensor 11 attached to a free face of a micro-Peltier component 6.

Advantageously, the temperature sensor 11 is a thermocouple having a diameter of 0.1 mm and accordingly, very low thermal inertia. The temperature sensor 11 is soldered onto the free face of a micro-Peltier component 6.

Naturally, the temperature sensor 11 is connected by any known means to the control and regulating circuit 3.

Advantageously, the thermal stimulation device conforming to the invention is associated with a computer 12 into which specific software is loaded for driving the control and regulating circuit 3.

Where applicable, various information or physical parameters can be transmitted directly by the stimulation head 2 to the computer 12 by any known type of communication link.

The stimulation head 2 and more particularly the micro-Peltier components 6 and the Peltier module 10 are connected through the control and regulation circuit 3 to an electric power supply source 13.

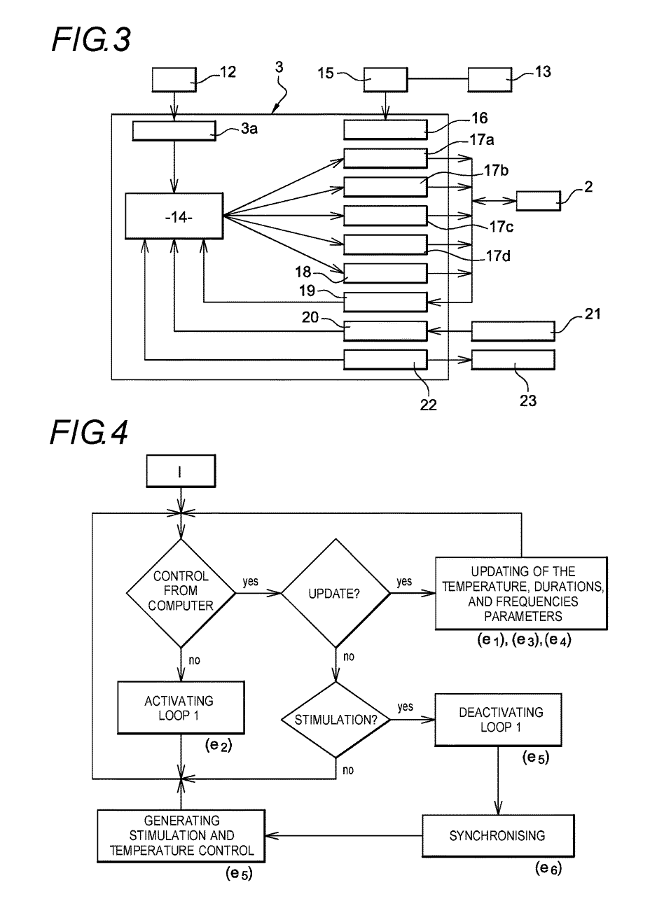

FIG. 3 is an example of the electronic architecture of the thermal stimulation device conforming to the invention.

The control and regulating circuit 3 includes a microcontroller 14 and a connection interface 3a to the computer 12.

Advantageously, the electric power supply source 13 can charge a battery 15 ensuring the independent operation of the thermal stimulation device.

Advantageously, the battery 15 and the control and regulating circuit 3 are incorporated in a unit 3b. The latter, for instance, is not integrated into the stimulation head 2.

In another example of an embodiment conforming to the invention, the thermal stimulation device can comprise super-capacitors accumulating energy between two cold stimulations, being supplied with power by a USB bus connected to the computer 12.

The control and regulating circuit 3, through battery 15, supplies electric voltage of approximately 40V to drive the micro-Peltier components 6 and the voltage/current regulating circuit of the said micro-Peltier components 6. Each row 6a, 6b, 6c, 6d, comprising four micro-Peltier components 6 connected in series is powered independently by electric voltage of 32 V. This limits the maximum voltage occurring at the thermal stimulation head 2 while providing for the safety of people in the event of a fault occurring in the electric insulation.

The control and regulating circuit 3 also provides a secondary electric power supply 16, producing approximately 5 Volts from battery 15 to supply the logic electronic device of the thermal stimulating device on the one hand and the Peltier module 10 on the other.

The secondary electric power supply 16 is produced advantageously using first a “step down” circuit integrated into the control and regulating circuit 3 and also the electric voltage supplied by battery 15.

The microcontroller 14 interprets the instructions received via connection interface 3a of the USB interface type and accordingly controls four current regulators 17a, 17b, 17c, 17d supplying respectively the rows 6a, 6b, 6c, 6d of the micro-Peltier components 6.

This generates the cold thermal stimulation at the end of the stimulation head 2.

Microcontroller 14 controls a complementary current regulator 18 which drives the Peltier modules 10 to ensure the stability of the neutral temperature at the thermal inertia mass 5.

Microcontroller 14 also drives the thermal stimulation device by means of temperature measurements 19 from temperature sensor 11 and, where applicable, by means of complementary measures 20 from complementary temperature sensor 21 placed directly on the tissue 1. The use of a complementary temperature sensor 21 like this facilitates the continuous adjustment of the neutral temperature to the tissue 1 temperature.

For instance, microcontroller 14 also drives triggering device 22 to generate a triggering signal designed to synchronise a physiological parameter recorded 23 with the cold thermal stimulations.

The electric supply of the micro-Peltier components 6 is therefore controlled to set up the hot faces on the thermal inertia mass 5. The control and regulating circuit 3 therefore allows a regulating loop to be performed to generate the cold stimulation temperature at the end of stimulation head 2.

When it becomes necessary to generate hot thermal stimulations, it simply means modifying the direction of the electric current conducted through the micro-Peltier components 6.

The electric supply to the Peltier module 10 is controlled so that its hot face is in contact with the thermal dissipator 9 and its cold face is in contact with the thermal inertia mass 5. The control and regulating circuit 3 thus allows another final regulating loop to be performed, maintaining the thermal inertia mass 5 at a determined temperature referred to as the neutral temperature. This neutral temperature corresponds, for instance, to the temperature of tissue 1.

This invention also refers to a test and control process for a cold thermal stimulating device. The test and control method consists in determining a neutral temperature applied to tissue 1. This neutral temperature corresponds, for instance, to the ambient temperature and/or the temperature of the tissue 1 to be explored.

Advantageously, the complementary temperature sensor 21 placed directly on the said tissue 1, facilitates the adjustment of the neutral temperature. The latter is set up very quickly through the end layer 18, considering its thinness, its low thermal inertia and its high thermal conductivity.

The process then consists in carrying out thermal regulation of the neutral temperature by activating a regulating loop. This loop is performed by means of microcontroller 14 controlling the electric power supply of Peltier module 10. The regulation loop is activated by default if no cold stimulation control is issued by computer 12.

The test and control method then comprises the determination of a cold thermal stimulation temperature to be applied to the tissue 1 in alternation with the neutral temperature. A control from computer 12 then generates information requiring possible updating of the temperature, duration and frequency parameters for the cold stimulations. It is then necessary to determine, modify or confirm a cold temperature, a duration and a frequency for the cold thermal stimulations that will be applied to tissue 1.

If an instruction is given to initiate cold or hot stimulation, one carries out regulation of the cold thermal stimulation temperature by activating a regulating loop and deactivating the regulating loop controlling the neutral temperature.

The regulation of the cold stimulation temperature is obtained by microcontroller 14 driving the electric power supplies of the micro-Peltier components 6 and more specifically, each row 6a, 6b, 6c, 6d of the micro-Peltier components 6.

When the cold thermal stimulation is completed, the neutral temperature thermal regulating loop is reactivated to re-establish the neutral temperature until the next cold thermal stimulation occurs. The use of the complementary temperature sensor 21 arranged advantageously near the stimulation zone allows finer adjustment of the neutral temperature.

Since the two regulating loops use the same temperature sensor or 11, the regulating loop on the neutral temperature has to be deactivated during the cold stimulation phases. During cold stimulation, the temperature sensor 11 reads the cold temperature resulting in ordering the heating of thermal inertia mass 5 if the neutral temperature regulating loop had remained active. Overall thermal perturbation would then be inevitable.

In another example of an embodiment conforming to the invention, the thermal stimulation device could comprise a specific temperature sensor reading the temperature of the thermal inertia mass 5. In this case, there would be no need to deactivate the regulation loop for the neutral temperature during the cold stimulation phases.

The test and control method conforming to the invention would also comprise the synchronising of the initiation of a physiological parameter recording read on the tissue 1, after the initiation of cold thermal stimulation.

Implementing the test and control method conforming to the invention consists in using the thermal inertia mass 5 to evacuate the thermal energy given off by the micro-Peltier components 6 during the cold thermal stimulation phases and in using the Peltier module 10 associated with the thermal dissipator 9 to evacuate the thermal energy from the said thermal inertia mass 5.

The Peltier module 10 can therefore heat the inertia mass 5. This could be the case when at ambient temperature the stimulation device is started at around 25° C. and the temperature of the thermal inertia mass 5 has to be brought to a neutral temperature, for instance a temperature included between 30° C. and 34° C. Similarly, it may be necessary to heat the thermal inertia mass 5 to maintain the neutral temperature and thus compensate for thermal exchanges with the ambient air.

The adding of thermal energy to the thermal inertia mass 5 may also be necessary during hot thermal stimulations during which the micro-Peltier components 6 take thermal energy from the said thermal inertia mass 5.

The implementing of the method conforming to the invention then allows, without any structural modification to the device, the generation either of cold thermal stimulations or hot thermal stimulations.

The test and control method is then implemented by means of control codes, integrated into a program loaded into computer 12, specific to the various nerve fibre investigation protocols.

It is obvious that this description is not confined to the examples explicitly described but also extends to other embodiments and/or implementation methods. Accordingly, another technical characteristic described here can be replaced by an equivalent technical characteristic and a described process step can be replaced by an equivalent step while remaining in the context of this invention.

1. A device for cold and/or hot thermal stimulation of an animal or human tissue, comprising:
a stimulation head designed to come into contact with said tissue,
a thermal control and regulating circuit for generating a stimulation temperature,
a thermal inertia mass comprising an end face,
micro-Peltier components integral with the end face of the thermal inertia mass to form an end in contact with the tissue, the micro-Peltier components comprising hot and cold faces,
a thermal dissipator,
at least one Peltier module sandwiched between the inertia mass and the thermal dissipator,
at least one temperature sensor attached to a free face of a micro-Peltier component, the sensor being connected to the thermal control and regulating circuit,
an electric power supply for the micro-Peltier components controlled by the thermal control and regulating circuit to produce the hot and/or cold faces in contact with the thermal inertia mass, the control and regulating circuit thus allowing a thermal stimulation regulating loop to be performed to generate the cold and/or hot stimulation temperature, and
an electric power supply for the Peltier module controlled by the control and regulating circuit to arrange its hot face in contact with the thermal dissipator or in contact with the thermal inertia mass, thus allowing a neutral temperature regulating loop for maintaining the thermal inertia mass at a neutral temperature by evacuating the thermal energy of the thermal inertia mass during a cold thermal stimulation phase or by compensating for the thermal energy drawn off during a hot thermal stimulation phase.

2. The device according to claim 1, wherein the temperature sensor is a thermocouple.

3. The device according to claim 1, wherein the control and regulating circuit comprises a microcontroller, the microcontroller comprising a connection interface for a computer and another connection interface for a physiological parameter recorder.

4. The device according to claim 1, wherein the micro-Peltier components, separate from each other and arranged in an array, are soldered to the end face of the thermal inertia mass, and wherein the device further comprises an electrical and thermal insulating material filling the free spaces located between the micro-Peltier components.

5. The device according to claim 1, further comprising an additional temperature sensor configured to continuously read the temperature of the thermal inertia mass.

6. A method for testing and controlling the device of claim 1, the method comprising:
determining the neutral temperature to be applied to the tissue,
carrying out thermal regulation of the neutral temperature by activating the neutral temperature regulating loop,
determining the cold and/or hot thermal stimulation temperature to be applied to the tissue in alternation with the neutral temperature,
determining a duration and a frequency for the application of the cold and/or hot thermal stimulation temperature,
if an instruction is given to apply the cold and/or hot thermal stimulation temperature, regulating the cold and/or hot thermal stimulation temperature by activating a thermal stimulation regulating loop and deactivating the neutral temperature regulating loop,
synchronising the triggering of a physiological parameter recorder with the application of the cold and/or hot thermal stimulation temperature, and
reactivating the neutral temperature regulating loop after each application of the cold and/or hot thermal stimulation temperature.

7. The method of claim 5, the method comprising:
determining a neutral temperature to be applied to the tissue,
carrying out thermal regulation of the neutral temperature by activating a neutral temperature regulating loop,
determining a cold and/or hot thermal stimulation temperature to be applied to the tissue in alternation with the neutral temperature,
determining a duration and a frequency for application of the cold and/or hot thermal stimulation temperature,
if an instruction is given to apply the cold and/or hot stimulation temperature, regulating the cold and/or hot thermal stimulation temperature by activating a thermal stimulation regulating loop, and
synchronising the triggering of a physiological parameter recorder with the application of the cold and/or hot thermal stimulation temperature.

8. The method of claim 7, further comprising continuously reading the temperature of the thermal inertia mass with the additional temperature sensor.

9. The method of claim 6, further comprising using micro-Peltier components to generate the cold and/or hot thermal stimulation temperature.

10. The method of claim 6, further comprising using a thermal inertia mass and a thermal dissipator associated with a Peltier module to generate the neutral temperature.

11. The method of claim 9, further comprising using the thermal inertia mass to evacuate thermal energy given off by the micro-Peltier components during the cold thermal stimulation phases.

12. The method of claim 9, further comprising using the thermal inertia mass to compensate the thermal energy drawn off by the micro-Peltier components during the hot thermal stimulation phases.

13. The method of claim 6, further comprising measuring the temperature of the tissue, at a determined frequency, and using the results of the measurement to determine or adjust the neutral temperature.

14. The method of claim 7, further comprising using micro-Peltier components to generate the cold and/or hot thermal stimulation temperature.

15. The method of claim 14, further comprising using the thermal inertia mass to evacuate thermal energy given off by the micro-Peltier components during the cold thermal stimulation phases.

16. The method of claim 14, further comprising using the thermal inertia mass to compensate the thermal energy drawn off by the micro-Peltier components during the hot thermal stimulation phases.

17. The method of claim 7, further comprising using a thermal inertia mass and a thermal dissipator associated with a Peltier module to generate the neutral temperature.

18. The method of claim 7, further comprising measuring the temperature of the tissue, at a determined frequency, and using the results of the measurement to determine or adjust the neutral temperature.

19. The method of claim 6, further comprising continuously reading the temperature of the thermal inertia mass with an additional temperature sensor.
